(19) United States
(12) Patent Application Publication
(10) Pub. No.: US 2004/0208837 A1
(43) Pub. Date: Oct. 21, 2004

(54) Agents and method for treating keratinous fibers

(76) Inventors: Marcus Krueger, Hamburg (DE); Elisabeth Poppe, Hamburg (DE)

Correspondence Address: HENKEL CORPORATION, THE TRIAD, SUITE 200, 2200 RENAISSANCE BLVD., GULPH MILLS, PA, 19406, US

(21) Appl. No.: 10/769,264

(22) Filed: Jan. 30, 2004

Related U.S. Application Data
(62) Division of application No. 09/623,352, filed on Sep. 1, 2000, now abandoned.
Application No. 09/623,352 is a 371 of international application No. PCT/EP99/01109, filed on Feb. 20, 1999.

(30) Foreign Application Priority Data
Mar. 2, 1998 (DE) 198 08 766.7

(51) Int. Cl. A61K 7/42

(52) U.S. Cl. 424/59

(57) ABSTRACT

Compositions and processes for treating keratin fibers. The compositions contain UV-absorbing cationic substances formulated as PIT microemulsions. The UV-absorbing substance consists of a UV-absorbing group and at least one quaternary ammonium component.

This invention relates to a composition in the form of a micro-emulsion for treating keratin fibers and to a process for treating keratin fibers using this composition.

Nowadays, human hair is treated in many different ways with hair-care preparations. Such treatments include, for example, the cleaning of hair with shampoos, the care and regeneration of hair with rinses and conditioners and the bleaching, coloring and shaping of hair with bleaching, coloring and tinting formulations, wave formulations and styling preparations. Apart from the actual purpose served by these products, efforts are increasingly being made to achieve preventive protection against damage with these preparations. Growing significance attaches in this regard to protection against the effects of excessive exposure to light which can occur, in particular, in the mountains and at sea through the UV component of solar radiation.

The use of compositions containing inorganic pigments or organic UV filters, for example based on benzophenone or cinnamic acid, for protecting skin and hair against solar radiation is known. Unfortunately, one of the disadvantages of such compositions, particularly where they are formulated as rinse-off products, lies in the poor substantivity of the active ingredients on the hair. Accordingly, the protective effect obtained with these products is generally unsatisfactory or not entirely satisfactory.

An attempt to overcome this disadvantage consisted in the development of new derivatives of the known filters which contain a cationic group in addition to the UV-absorbing component. The substantivity of these substances on the hair is thus distinctly improved. Unfortunately, the uneven distribution of the adsorbed filters on the hair does present problems. Although these problems can be solved by adding fatty or oil components to the corresponding compositions, problems can then arise in regard to the formulation of storage-stable products and, in the particular case of products formulated as sprays, acceptable spraying behavior can only be achieved by the simultaneous addition of organic solvents. However, the use of such solvents is not only undesirable for ecological reasons, it can also have an adverse effect on the hair-care properties of the compositions.

It has now been found that the problems mentioned above can be overcome by using special UV-absorbing cationic substances in compositions formulated as special microemulsions, so-called PIT emulsions.

It was known to the expert that the introduction of ionic components into PIT emulsions normally leads to a considerable increase in the phase inversion temperature. Accordingly, the expert will generally refrain from preparing such products either for substance-specific reasons or for economic reasons. This applies in particular to the quaternary ammonium compounds known as conditioners which, in the quantities normally used, often produce an increase of 20 to 30° C. in the phase inversion temperature to beyond the critical limit of 100° C.

Accordingly, it is extremely surprising that the UV-absorbing substances containing cationic groups used in accordance with the invention either do not measurably influence the phase inversion temperature at all or increase it only slightly, i.e. generally well below 15° C.

Accordingly, the present invention relates to compositions for treating keratin fibers which contain at least 6% by weight of oil components and at least one nonionic emulsifier and which are present as a microemulsion with a mean droplet diameter of less than 400 nanometers and which, in addition, contain a UV-absorbing component A corresponding to general formula (I):

U−Q (I)

in which U is a UV-absorbing group and Q is a group which contains at least one quaternary ammonium function.

In the context of the invention, keratin fibers are understood to be pelts, wool, feathers and, in particular, human hair.

The compositions according to the invention contain an oil component as a first compulsory constituent. Suitable oil components are, in principle, any water-insoluble oils and fatty compounds and mixtures thereof with solid paraffins and waxes. In the context of the invention, water-insoluble substances are understood to be substances with a solubility in water at 20° C. of less than 0.1% by weight. The melting point of the individual oil or fatty components should be below the phase inversion temperature of the system and, more particularly, below about 40° C. According to the invention, oil and fatty components which are liquid at room temperature, i.e. below 25° C., can be particularly preferred. Where several oil and fatty components and optionally solid paraffins and waxes are used, however, it is generally sufficient if the mixture of the oil and fatty components and optionally paraffins and waxes satisfy these conditions.

A preferred group of oil components are vegetable oils. Examples of such oils are sunflower oil, olive oil, soybean oil, rapeseed oil, almond oil, jojoba oil, orange oil, wheat germ oil, peach kernel oil and the liquid components of coconut oil.

However, other triglyceride oils, such as the liquid components of bovine tallow, and synthetic triglyceride oils are also suitable.

Another group of compounds suitable for use as oil components in accordance with the invention are liquid paraffin oils and synthetic hydrocarbons and di-n-alkyl ethers containing a total of 12 to 36 carbon atoms and, more particularly, 12 to 24 carbon atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and ditert.butyl ether, diisopentyl ether, di-3-ethyldecyl ether, tert.butyl-n-octyl ether, isopentyl-n-octyl ether and 2-methylphenyl-n-octyl ether. The compounds 1,3-di-(2-ethylhexyl)-cyclohexane and di-n-octyl ether obtainable as commercial products (Cetiol® S and Cetiol®) OE, respectively) can be preferred.

Other oil components suitable for use in accordance with the invention are fatty acid and fatty alcohol esters. The monoesters of fatty acids with alcohols containing 3 to 24 carbon atoms are preferred. This group of substances are products of the esterification of fatty acids containing 8 to 24 carbon atoms such as, for example, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural fats and oils, in the reduction of aldehydes from Roelen's oxosynthesis or in the dimerization of unsaturated fatty acids with alcohols such as, for example isopropyl alcohol, caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxosynthesis and as monomer fraction in the dimerization of unsaturated fatty alcohols. According to the invention, isopropyl myristate, isononanoic acid C₁₆₋₁₈ alkyl ester (Cetiol® SN), stearic acid-2-ethylhexyl ester (Cetiol® 868), cetyl oleate, glycerol tricaprylate, cocofatty alcohol caprate/caprylate and n-butyl stearate are particularly preferred.

Finally, other oil components suitable for use in accordance with the invention are dicarboxylic acid esters such as di-n-butyl adipate, di-(2-ethylhexyl)-adipate, di-(2-ethylhexyl)-succinate and diisotridecyl acelate, and diol esters, such as ethylene glycol dioleate, ethylene glycol diisotridecanoate, propylene glycol di-(2-ethylhexanoate), propylene diisostearate, propylene glycol dipelargonate, butanediol diisostearate and neopentyl glycol dicaprylate.

The total quantity of oil and fatty components in the compositions according to the invention is normally from 6 to 45% by weight, based on the composition as a whole. According to the invention, quantities of 10 to 35% by weight are preferred.

The compositions according to the invention contain a nonionic emulsifier as a secondary compulsory constituent.

Suitable nonionic emulsifiers are, for example,

products of the addition of 4 to 30 moles ethylene oxide and/or 0 to 5 moles propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of addition products of 1 to 30 moles ethylene oxide onto polyols containing 3 to 6 carbon atoms, more particularly glycerol,

ethylene oxide and polyglycerol addition products with methyl glucoside fatty acid esters, fatty acid alkanolamides and fatty acid glucamides,

C₈₋₂₂ alkyl monoglycosides and oligoglycosides and ethoxylated analogs thereof, degrees of oligomerization of 1.1 to 5 and more particularly 1.2 to 1.4 and glucose as the sugar component being preferred,

products of the addition of 5 to 60 moles ethylene oxide onto castor oil and hydrogenated castor oil and

partial esters of polyols containing 3 to 6 carbon atoms with saturated fatty acids containing 8 to 22 carbon atoms.

The compositions according to the invention contain the nonionic emulsifiers in quantities of preferably 4 to 15% by weight and more preferably 6 to 10% by weight, based on the composition as a whole.

The compositions according to the invention preferably contain at least one nonionic emulsifier with an HLB value of 8 to 18 as defined in Römpp—Lexikon Chemie (eds. J. Falbe, M. Regitz), 10th Edition, Georg Thieme Verlag, Stuttgart/New York (1997), page 1764. According to the invention, nonionic emulsifiers with an HLB value of 10 to 15 can be particularly preferred.

Among the nonionic emulsifiers mentioned, ethoxylated fatty alcohols containing 8 to 22 carbon atoms and 4 to 30 EO units are preferred.

Another compulsory component of the compositions according to the invention are special UV-absorbing components A which are also referred to in short hereinafter as UV absorbers A. These UV absorbers A have the general structure UΣQ.

The structural moiety U stands for a UV absorbing group. In principle, this group may be derived from the known UV filters used in cosmetics in which a group, generally a hydrogen atom, of the UV filter is replaced by a group Q with a quaternary amino function. The following are examples of compounds from which the structural moiety U may be derived:

substituted benzophenones, such as 2,4-dihydroxybenzophenone, 2,2′,4,4′-tetrahydroxybenzophenone, 2-hydroxy-4-methoxybenzophenone, 2,2′-dihydroxy-4,4′-dimethoxybenzophenone, 2-hydroxy-4-methoxybenzophenone-5-sulfonic acid and 2,2′-dihydroxy-4,4′-dimethoxy-benzophenone-5-sodium sulfonate,

p-aminobenzoic acid ester,

diphenyl acrylates, such as ethyl-2-cyano-3,3-diphenyl acrylate and 2′-ethylhexyl-2-cyano-3,3-diphenyl acrylate,

cinnamic acid esters, such as octyl methoxycinnamate,

salicylic acid esters, such as octyl salicylate,

benzimidazoles and

o-aminobenzoic acid esters.

According to the invention, structural moieties U derived from cinnamic acid amide or from N,N-dimethylaminobenzoic acid amide are preferred.

In principle, the structural moieties U may be selected so that the absorption maximum of the UV absorber A can lie both in the UVA (315-400 nm) and in the UVB (280-315 nm) range or in the UVC (<280 nm) range. UV absorbers A with an absorption maximum in the UVB range, more particularly in the range from about 280 to about 300 nm, are particularly preferred.

In addition, the structural moiety U—again in dependence on the structural moiety Q—is preferably selected so that the molar extinction coefficient of the UV absorber A at the absorption maximum is above 15,000 and more particularly above 20,000.

The structural moiety Q must contain a quaternary ammonium group. In principle, this quaternary ammonium group may be directly attached to the structural moiety U, so that the structural moiety U represents one of the four substituents of the positively charged nitrogen atom. However, one of the four substituents at the positively charged nitrogen atom is preferably a group, more particularly an alkylene group containing 2 to 6 carbon atoms, which acts as a link between the structural moiety U and the positively charged nitrogen atom.

In one preferred embodiment of the invention, Q has the general structure —(CH₂)ₓ—N⁺R¹R²R³ X [symbol] , in which x is an integer of 1 to 4, R¹ and R² independently of one another represent C₁₋₄ alkyl groups, R³ is a C₁₋₂₂ alkyl group or a benzyl group and X [symbol] is a physiologically compatible anion. In the general structure indicated above, x is preferably the number 3, R¹ and R² each represent a methyl group and R³ is either a methyl group or a saturated or unsaturated, linear or branched hydrocarbon chain containing 8 to 22 and more particularly 10 to 18 carbon atoms.

Physiologically compatible anions are, for example, inorganic anions, such as halides, more particularly chloride, bromide and fluoride, sulfate ions and phosphate ions and organic anions, such as lactate, citrate, acetate, tartrate, methosulfate and tosylate.

Two particularly preferred UV absorbers A are the compounds cinnamic acid amidopropyl trimethyl ammonium chloride and dodecyl dimethyl aminobenzamidopropyl dimethyl ammonium tosylate obtainable as commercial products (Incroquat®UV-283 and Escalol® HP 610, respectively).

The UV absorbers A are present in the compositions according to the invention in quantities of normally 0.5 to 5% by weight and preferably 1.0 to 2.5% by weight, based on the composition as a whole.

Water is another compulsory component of the compositions according to the invention.

Finally, the compositions according to the invention must be present in the form of a microemulsion with a mean droplet diameter of less than 400 nm and, more particularly, less than 200 nm.

Microemulsions in the context of the present invention are understood to be so-called “PIT” emulsions. These emulsions are, in principle, systems containing the three components water, oil and nonionic emulsifier which are present as an oil-in-water (o/w) emulsion at room temperature. When these systems are heated, microemulsions are formed in a certain temperature range (normally referred to as the phase inversion temperature or “PIT”) and, on continued heating, change into water-in-oil (w/o) emulsions. In the event of subsequent cooling, o/w emulsions are reformed, but are present even at room temperature as microemulsions with a mean particle diameter of less than 400 nm and, more particularly, with a particle diameter of about 100 to 300 nm. Particulars of these very stable low-viscosity systems, which are now generally known as “PIT emulsions”, can be found in a number of publications, of which the articles in Angew. Chem. 97, 655-669 (1985) and Adv. Colloid Interface Sci. 58, 119-149 (1995) are representative.

According to the invention, microemulsions or PIT emulsions with a mean particle diameter of around 200 nm can be preferred.

The microemulsions according to the invention may be prepared, for example, by initially determining the phase inversion temperature of the system by heating a sample of the conventionally prepared emulsion and determining the temperature at which there is a drastic reduction in conductivity using a conductimeter. The reduction in the specific conductivity of the o/w emulsion initially present generally decreases from originally more than 1 mS/cm to values below 0.1 mS/cm over a temperature range of 2 to 8° C. This temperature range thus corresponds to the phase inversion temperature range. Accordingly, once the phase inversion temperature is known, the emulsion of oil component, nonionic emulsifier, at least parts of the water and optionally other components initially prepared in the usual way may be heated to a temperature within or above the phase inversion temperature range and subsequently cooled and other components, if any, and the remaining water may be added. Alternatively, the microemulsion may also be directly prepared at a temperature lying within or above the phase inversion temperature range. The microemulsion thus prepared is then cooled to a temperature below the phase inversion temperature range, normally room temperature.

Besides the compulsory components, the compositions according to the invention may contain other components, depending upon the particular application envisaged, of which the influence on the phase inversion temperature can have a crucial bearing on the usability of individual compound.

In one preferred embodiment, the compositions according to the invention are characterized in that, in addition to the oil components which also have a hair-care effect in many cases, they contain at least one other hair-care component. This other hair-care component is generally selected from nonionic compounds. In principle, however, this does not preclude the use of ionic hair-care compounds, particularly if they are employed in small quantities. In this case, however, the influence of these ionic compounds on the phase inversion temperature should be determined. In general, only those ionic components whose influence on the phase inversion temperature does not pose any additional problems in the production process will generally be used.

Active substances with a hair-care effect are known to the expert. Examples include

Fatty alcohols containing 8 to 22 carbon atoms. The fatty alcohols used may be saturated or unsaturated and linear or branched. Fatty alcohols suitable for use in accordance with the invention are, for example, decanol, octanol, octenol, dodecenol, decenol, octadienol, dodecadienol, decadienol, oleyl alcohol, erucyl alcohol, ricinolyl alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol and their Guerbet alcohols, this list being intended to be purely exemplary without any limiting character. However, the fatty alcohols emanate from preferably natural fatty acids and are normally produced from the esters of the fatty acids by reduction. Also suitable for use in accordance with the invention are fatty alcohol cuts which are produced by reduction of naturally occurring triglycerides, such as bovine tallow, palm oil, peanut oil, rapeseed oil, cottonseed oil, soybean oil, sunflower oil and linseed oil or fatty acid esters formed from transesterification products thereof with corresponding alcohols and which therefore represent a mixture of different fatty alcohols. The fatty alcohols are preferably used in quantities of 0.3 to 3% by weight, based on the preparation as a whole.

Animal and preferably vegetable protein hydrolyzates such as, in particular, elastin, collagen, keratin, milk protein, soya protein, silk protein, oat protein, pea protein, almond protein and wheat protein hydrolyzates, condensation products thereof with fatty acids and quaternized protein hydrolyzates.

Vitamins and vitamin precursors, such as tocopherols, vitamin A, nicotinic acid and nicotinic acid amide, other vitamins of the B complex, vitamin F and in particular biotin. Other preferred representatives of this group of hair-care substances are panthenol, derivatives thereof, more particularly esters and ethers of panthenol and cationically derivatized panthenols. Individual representatives are, for example, panthenol triacetate, panthenol monoethyl ether and its monoacetate and the cationic panthenol derivative disclosed in WO 92/13829. Within this group, panthenol is preferred.

Mono-, di- and oligosaccharides such as, for example, glucose, galactose, fructose, mannose, fruit sugar and lactose.

Plant extracts which are normally prepared by extracting the entire plant, but in some cases also exclusively from blossoms and/or leaves of the plant. So far as the plant extracts suitable for use in accordance with the invention are concerned, particular reference is made to the extracts listed in the Table beginning on page 44 of the 3rd Edition of the Leiffaden zur Inhaltsstoffdeklaration kosmetischer Mittel, published by the Industrieverband Körperpflege-und Waschmittel e.V. (IKW), Frankfurt. According to the invention, particular preference is attributed above all to the extracts of oak bark, stinging neftle, hamamelis, hops, camomile, burdock root, horse willow, hawthorn, lime blossom, almond, aloe Vera, pine needle, horse chestnut, sandalwood, juniper, coconut, mango, apricot, lemon, wheat, kiwi, melon, orange, grapefruit, sage, rosemary, birch, mallow, lady's smock, creeping thyme, yarrow, thyme, balm, restharrow, coltsfoot, hibiscus, meristem, ginseng and ginger root. The extracts of almond, aloe vera, coconut, mango, apricot, lemon, wheat, kiwi and melon are particularly preferred. The compositions according to the invention may also contain mixtures of several, more particularly two, different plant extracts. Water, alcohols and mixtures thereof may be used as extractants for preparing the plant extracts mentioned. Among the alcohols, lower alcohols, such as ethanol and isopropanol, but especially polyhydric alcohols, such as ethylene glycol, propylene glycol and butylene glycol, are particularly preferred both as sole extractant and in admixture with water. Plant extracts based on water/propylene glycol in a ratio of 1:10 to 10:1 have proved to be particularly suitable. According to the invention, the plant extracts may be used both in pure form and in dilute form. If they are used in dilute form, they normally contain around 2 to 80% by weight of active substance and—as solvent—the extractant or extractant mixture used in their production.

Honey extracts which are obtained similarly to the plant extracts and which normally contain 1 to 10% by weight and more particularly 3 to 5% by weight of active substance. In this case, too, water/propylene glycol mixtures can be preferred extractants.

Ceramides.

Phospholipids, for example soya lecithin, egg lecithin and kephalins.

Silicone oils, more particularly dialkyl and alkylaryl siloxanes such as, for example, dimethyl polysiloxane and methylphenyl polysiloxane and alkoxylated and quaternized analogs thereof. Examples of such silicones are the products marketed by Dow Corning under the names of DC 190, DC 200 and DC 1401 and the Dow Corning products DC 344 and DC 345, Q2-7224 (manufacturer: Dow Corning, a stabilized trimethyl silyl amodimethicone), Dow Corning® 929 Emulsion (containing a hydroxylamino-modified silicone which is also known as Amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

Alkylamidoamines which are normally obtained by amidation of natural or synthetic fatty acids and fatty acid cuts with dialkylaminoamines. Typical examples of such fatty acids are caproic acid, caprylic acid, 2-ethylhexyanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural and oils, in the reduction of aldehydes from Roelen's oxosynthesis or in the dimerization of unsaturated fatty acids. Normally, the fatty acid cuts obtainable from coconut oil or palm oil are particularly preferred; in general, the use of stearic acid is particularly preferred. According to the invention, a particularly suitable compound from this group of substances is the stearamidopropyl dimethyl amine commercially obtainable as Tegoamid® S 18.

Esterquats, more particularly quaternized fatty acid triethanolamine ester salts, quaternized ester salts of fatty acids with diethanol alkylamines and quaternized ester salts of fatty acids with 1,2-dihydroxypropyl dialkylamines.

Cationic surfactants of the quaternary ammonium compound type, more particularly ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride, tricetyl methyl ammonium chloride, behenyl tri-methyl ammonium methosulfate and the imidazolinium compounds known by the INCI names of Quaternium-27 and Quaternium-83.

Cationic polymers, more particularly those containing a quaternary nitrogen atom, for example in the form of an ammonium group. Preferred cationic polymers are, for example, the quaternized cellulose derivatives commercially obtainable under the names of Celquat® and Polymer JR® (more particularly the products Celquat® H 100, Celquat® L 200 and Polymer JR® 400), polymeric dimethyl diallyl ammonium salts and copolymers thereof with esters and amides of acrylic acid and methacrylic acid which are commercially obtainable under the names of Merquat® 100 (poly(dimethyl diallyl ammonium chloride)) and Merquat® 550 (dimethyl diallyl ammonium chloride/acrylamide copolymer), copolymers of vinyl pyrrolidone with quaternized derivatives of dialkylaminoacrylate and methacrylate, such as for example diethyl sulfate-quaternized vinyl pyrrolidone/dimethyl aminoalkyl methacrylate copolymers (for example the commercial products Gafquat® 734 and Gafquat® 755), vinyl pyrrolidone/vinyl imidazolinium methochloride copolymers commercially obtainable under the name of Luviquate, quaternized polyvinyl alcohol and the polymers with quaternary nitrogen atoms in the main polymer chain known by the names of Polyquaternium 2, Polyquaternium 17, Polyquaternium 18 and Polyquaternium 27.

Particulars of other compounds can be found in the reference books known to the expert, for example K. Schrader, Grundlagen und Rezepturen der Kosmetika 2nd Edition, Hüthig Buch Verlag, Heidelberg, 1989.

The additional hair-components are present in the compositions according to the invention in quantities of preferably 0.05 to 10% by weight and more preferably 0.1 to 5% by weight, based on the active substance of the hair-care component and the composition as a whole.

In another preferred embodiment, the compositions according to the invention contain another UV filter in addition to the UV absorber A. The other UV filter may be selected from any of the standard UV filters suitable for use in cosmetics. According to the invention, oil-soluble UV filters and substances with an absorption maximum in the UVB range can be preferred. So far as the other UV filters generally used are concerned, reference is made to the foregoing observations. Preferred other UV filters are 2-ethylhexyl-3,3′-diphenyl-2-cyanoacrylate (OCTOCRYLENE), 2-ethylhexyl-4-methoxycinnamate (OCTYLMETHOXYCINNAMATE), 2-hydroxy-4-methoxybenzophenone (BENZOPHENONE-3), isoamyl-4-methoxycinnamate (ISOAMYL P-METHOXYCINNAMATE) and 2-phenyl benzimidazole-5-sulfonic acid (PHENYL BENZIMIDAZOLE SULFONIC ACID). These other UV filters are present in the compositions according to the invention in a quantity of preferably 0.1 to 7% by weight and more preferably 0.2 to 5% by weight, based on the composition as a whole.

In a first embodiment, the compositions according to the invention are formulated as a hair rinse or hair conditioner. Hair rinses are generally formulated in such a way that the active substances are rinsed out with water or with an at least predominantly water-containing preparation after the required contact time. The contact time with the hair is generally short. Hair conditioners contain the combination of active substances in a higher concentration than hair rinses and are intended for the treatment of seriously damaged hair. The contact time can be short, for example of the same order as the contact time of hair rinses, although it may even be as long as 20 minutes, depending on the degree of hair damage. The hair conditioners according to the invention may be rinsed out with water or with an at least predominantly water-containing preparation after that contact time. However, they may also be left on the hair. These compositions may advantageously be formulated as foam aerosols, but especially as sprays. To this end, the compositions contain propellant gases. In this variant, however, they are preferably formulated as pump sprays with air as the propellant.

In other embodiments, the compositions according to the invention may be, for example, cleaning compositions, such as shampoos, hair-setting compositions, such as lacquers, sprays and gels, permanent shaping formulations, such as permanent wave and fixing sets, color-changing formulations, such as blonding preparations, oxidation colorants and tints based on substantive dyes, hair lotions and hair tip fluids. The viscosities of the preparations may thus be adjusted according to the particular application envisaged. In addition, the invention also encompasses products which, as separately packed preparations, comprise on the one hand a composition according to the invention and in addition one other preparation comprising, for example, oxidation dye precursors or oxidizing agents. These separately packed preparations are then either mixed immediately before application to the hair or are combined on the hair itself.

Accordingly, other typical ingredients of the compositions according to the invention—always with the above-mentioned limitation in regard to the phase transition temperature range, particularly in the case of ionic compounds—may be the following:

anionic, zwitterionic, amphoteric and nonionic polymers such as, for example, vinyl acetate/crotonic acid copolymers, polydimethyl siloxanes, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers;

anionic surfactants such as, in particular, alkyl sulfates, alkyl polyglycolether sulfates and ether carboxylic acids containing 10 to 18 carbon atoms in the alkyl group and up to 12 glycol ether groups in the molecule and sulfosuccinic acid mono- and dialkyl esters containing 8 to 18 carbon atoms in the alkyl group and sulfosuccinic acid monoalkyl polyohydroxyethyl esters containing 8 to 18 carbon atoms in the alkyl group and 1 to 6 hydroxyethyl groups;

zwitterionic surfactants, more particularly the so-called betaines, such as the N-alkyl-N,N-dimethylammonium glycinates, for example cocoalkyl dimethylammonium glycinate, N-acylaminopropyl-N,N-dimethylammonium glycinates, for example cocoacylaminopropyl dimethyl ammonium glycinate, and 2-alkyl-3-carboxylmethyl-3-hydroxyethyl imidazolines containing 8 to 18 carbon atoms in the alkyl or acyl group and cocoacylaminoethyl hydroxyethyl carboxymethyl glycinate;

ampholytic surfactants, such as N-alkylglycines, N-alkylpropionic acid, N-alkylaminobutyric acid, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing 8 to 18 carbon atoms in the alkyl group;

defoamers, such as silicones;

thickeners, such as agar agar, guar gum, alginates, xanthan gum, gelatin, pectin, hydroxyethyl cellulose and also polyacrylamides and copolymers thereof;

structurants, such as maleic acid;

perfume oils, dimethyl isosorbide and cyclodextrins;

solubilizers, such as ethylene glycol, propylene glycol, glycerol and diethylene glycol;

dyes for coloring the composition;

antidandruff agents, such as piroctone olamine, zinc omadine and climbazol;

other substances for adjusting the pH;

active principles, such as allantoin, pyrrolidone carboxylic acids and bisabolol;

other UV filters;

consistency factors, such as sugar esters, polyol esters or polyol alkyl ethers;

fats and waxes, such as spermaceti, beeswax, montan wax and paraffins;

swelling and penetration agents, such as glycerol, propylene glycol monoethyl ether, carbonates, hydrogen carbonates, guanidines, ureas and primary, secondary and tertiary phosphates;

opacifiers, such as latex, styrene/PVP and styrene/acrylamide copolymers;

pearlizers, such as ethylene glycol monostearate and distearate and PEG-3-distearate;

complexing agents, such as EDTA, NTA, β-alanine diacetic acid and phosphonic acids;

substantive dyes;

so-called primary and secondary intermediates as oxidation dye precursors;

reducing agents such as, for example, thioglycolic acid and derivatives thereof, thiolactic acid, cysteamine, thiomalic acid and α-mercapto-ethane sulfonic acid;

oxidizing agents, such as hydrogen peroxide, potassium bromate and sodium bromate;

propellants, such as propane/butane mixtures, N₂O, dimethyl ether, CO₂, N₂ and air and

antioxidants.

In principle, the pH of the compositions according to the invention may be in the range from 2 to 11, the expert allowing for instabilities known to him, for example of the parent compound panthenol in the alkaline medium. However, the pH of the compositions according to the invention is preferably in the range from 2 to 7, values in the range from 3 to 6 being particularly preferred. This pH may be adjusted with virtually any acid suitable for cosmetic purposes. Edible acids are normally used. Edible acids are acids which are taken up as part of the normal food intake and which have positive effects on the human organism. Edible acids are, for example, acetic acid, lactic acid, tartaric acid, citric acid, malic acid, ascorbic acid and gluconic acid. According to the invention, citric acid and lactic acid are particularly preferred.

The present invention also relates to a process for treating keratin fibers, more particularly human hair, in which the composition claimed in any of claims 1 to 11 is applied to the fibers and is rinsed off again after a contact time of about 1 second to about 30 minutes.

The following Examples are intended to illustrate the invention.

### APPLICATION EXAMPLES

All quantities in the following Examples are parts by weight, unless otherwise indicated.

1. UV Protection Lotion

| | |
|---|---|
| (a) Cetiol ® SN¹ | 20.0 |
| (a) Cutina ® E 24² | 5.3 |
| (a) Cutina ® GMS³ | 3.6 |
| (a) Incroquat ® UV-283⁴ | 2.0 |
| (a) Neo Heliopan ® BB⁵ | 1.0 |
| (a) Perfume oil | 1.0 |
| (b) Panthenol | 0.2 |
| (b) Water, deionized | to 100 |

¹Isononanoic acid C₁₆₋₁₈ alkyl ester (INCI name: Cetearyl Isononanoate) (HENKEL)
²Glycerol monostearate + 24 EO (INCI name: PEG-20 Glyceryl Stearate)
³Glycerol monostearate (INCI name: Glyceryl Stearate) (HENKEL)
⁴Trimethyl cinnamic acid amidopropyl ammonium chloride (INCI name: Cinnamidopropyl Trimethyl Ammonium Chloride) (CRODA)
⁵2-Hydroxy-4-methoxybenzophenone (INCI name: Benzophenone-3 (HAARMANN & REIMER)

Preparation: two separate mixtures containing the components (a) and (b) are heated to 85° C. The two mixtures are then mixed at 85° C. and cooled with continuous stirring.

2. Sprayable Hair Conditioner, Leave-On

| | |
|---|---|
| (a) Monomuls ® 60-35 C⁶ | 1.7 |
| (a) Eumulgin ® B17 | 3.5 |
| (a) Cetiol ® S⁸ | 7.2 |
| (a) Cetiol ® OE⁹ | 7.2 |
| (a) Dow Corning ® 344-EU-Fluid¹⁰ | 3.6 |
| (a) Escalol ® HP610¹¹ | 1.5 |
| (a) Perfume oil | q.s. |
| (a) Preservative | q.s. |
| (b) Glycerol | 2.0 |
| (b) Water | to 100 |

⁶Fatty acid mono/diglyceride based on palm oil, hydrogenated (INCI name: Hydrogenated Palm Glycerides) (HENKEL)
⁷Cetyl stearyl alcohol + 12 EO (INCI name: Ceteareth-12) (HENKEL)
⁸1,3-Bis-(2-ethylhexyl)-cyclohexane (INCI name: Dioctylcyclohexane) (HENKEL)
⁹Di-n-octyl ether (INCI name: Dicaprylyl Ether) (HENKEL)
¹⁰Octamethyl cyclotetrasiloxane (INCI name: Cyclomethicone) (DOW CORNING)
¹¹Dodecyl dimethyl aminobenzamidopropyl dimethyl ammonium tosylate (ISP)

Preparation: as Example 1

3. Repair Lotion, Light

| | |
|---|---|
| (a) Emulgade ® SE¹² | 4.5 |
| (a) Eumulgin ® B2¹³ | 1.0 |
| (a) Cetiol ® LC¹⁴ | 5.0 |
| (a) Cetiol ® OE | 5.0 |
| (a) Incroquat ® UV-283 | 1.0 |
| (a) Preservative | q.s. |
| (b) Gluadin ® W 40¹⁵ | 0.5 |
| (b) Water, demineralized | to 100 |

¹²Mixture of partial glycerides, fatty alcohols, fatty alcohol ethoxylates and wax esters (INCI name: Glyceryl Stearate (and) Ceteareth-20 (and) Ceteareth-12 (and) Cetearyl Alcohol (and) Cetyl Palmitate) (HENKEL)
¹³Cetyl stearyl alcohol + 20 EO (INCI name: Ceteareth-20) (HENKEL)
¹⁴Caprylic acid/capric acid C₁₂₋₁₈ fatty alcohol ester (INCI name: Coco-Caprylate/Caprate) (HENKEL)
¹⁵Wheat protein hydrolyzate (40% active substance in water; INCI name: Aqua (and) Hydrolyzed Wheat Protein (and) Sodium Benzoate (and) Phenoxyethanol (and) Methylparaben (and) Propylparaben) (HENKEL)

Preparation: as Example 1

4. Repair Lotion, Sprayable

| | |
|---|---|
| (a) Emulgade ® SE | 4.5 |
| (a) Eumulgin ® B2 | 1.0 |
| (a) Cetiol ® LC | 5.0 |
| (a) Cetiol ® OE | 5.0 |
| (a) Escalol ® HP610 | 0.5 |
| (a) Preservative | q.s. |
| (b) Promois ® Silk 1000 Q¹⁶ | 0.5 |
| (b) Panthenol | 0.5 |
| (b) Water, demineralized | to 100 |

¹⁶Silk protein hydrolyzate (ca. 7% active substance in water; INCI name: Aqua (and) Hydrolyzed Silk) (INTERORGANA)

Preparation: as in Example 1

5. Hair Protection Lotion

| | |
|---|---|
| (a) Emulgade ® SE | 4.5 |
| (a) Eumulgin ® B2 | 1.0 |
| (a) Cetiol ® S | 5.0 |
| (a) Cetiol ® OE | 5.0 |
| (a) Escalol ® HP610 | 1.25 |
| (a) Preservative | q.s. |
| (a) Neo Heliopan ® BB | 0.75 |
| (b) Water | to 100 |

Preparation: as Example 1

6. Hair Protection Lotion, Forte

| | |
|---|---|
| (a) Emulgade ® SE | 4.5 |
| (a) Eumulgin ® B2 | 1.0 |
| (a) Cetiol ® S | 5.0 |
| (a) Cetiol ® OE | 5.0 |
| (a) Escalol ® HP610 | 2.0 |
| (a) Neo Heliopan ® BB | 1.5 |
| (a) Copherol ® F 1300¹⁷ | 0.3 |
| (b) Water | to 100 |

¹⁷D-α-tocopherol (85% active substance; INCI name: Tocopherol) (HENKEL CORP.)

Preparation as Example 1

7. Hair Repair Spray

| | |
|---|---|
| (a) Emulgade ® SE | 4.5 |
| (a) Eumulgin ® B2 | 1.0 |
| (a) Cetiol ® S | 5.0 |
| (a) Cetiol ® OE | 5.0 |
| (a) Incroquat ® UV-283 | 1.0 |
| (a) Copherol ® F 1300 | 0.2 |
| (b) Panthenol | 1.0 |
| (b) Water | to 100 |

¹⁷D-α-tocopherol (85% active substance; INCI name: Tocopherol) (HENKEL CORP.)

Preparation as Example 1

8. Sun Protection Balm

| | |
|---|---|
| (a) Emulgade ® SE | 1.6 |
| (a) Eumulgin ® B2 | 0.4 |
| (a) Cetiol ® OE | 5.5 |
| (a) Myritol ® 318¹⁸ | 0.5 |
| (a) Escalol ® HP 610 | 1.0 |
| (b) Neo Heliopan ® Hydro¹⁹ | 1.5 |
| (b) Hydagen ® B²⁰ | 1.0 |
| (b) Carbopol ® 5984²¹ (2% in water) | 8.0 |
| (b) NaOH, 1% | 4.0 |
| (b) Water, demineralized | to 100 |

¹⁸Fatty acid triglyceride (INCI name: Caprylic/Capric Triglyceride) (HENKEL)
¹⁹2-Phenylbenzimidazole-5-sulfonic acid (INCI name: Phenylbenz-imidazole Sulfonic Acid) (HAARMANN & REIMER)
²⁰D,L-α-bisabolol (85% active substance; INCI name: Bisabolol) (HENKEL)
²¹Polyacrylic acid (INCI name: Carbomer) (B. F. GOODRICH)

Preparation: as Example 1

9. Intensive Sun Protection Lotion (Leave-On)

| | |
|---|---|
| (a) Emulgade ® SE | 8.0 |
| (a) Eumulgin ® B2 | 2.0 |
| (a) Almond oil | 2.0 |
| (a) Eutanol ® G²² | 2.0 |
| (a) Cetiol ® 868²³ | 9.0 |
| (a) Incroquat ® UV-283 | 0.5 |
| (a) Neo Heliopan ® BB | 1.0 |
| (a) Neo Heliopan ® AV²⁴ | 1.0 |
| (b) Glycerol, 86% | 5.0 |
| (b) Water, demineralized | to 100 |

²²2-Octyl dodecyl alcohol (INCI name: OCTYLDODECANOL) (HENKEL)
²³Stearic acid-2-ethylhexyl ester (INCI name: Octyl Stearate) (HENKEL)
²⁴2-Ethylhexyl-4-methoxycinnamate (INCI name: Octyl Methoxycinnamate) (HAARMANN & REIMER)

Preparation: as Example 1

10. Sun Protection Emulsion, Sprayable

| | |
|---|---|
| (a) Emulgade ® SE | 4.7 |
| (a) Eumulgin ® B2 | 1.3 |
| (a) Cetiol ® 868 | 6.0 |
| (a) Cetiol ® Sn | 6.0 |
| (a) Escalol ® HP 610 | 2.0 |
| (a) Neo Heliopan ® E 1000²⁵ | 4.0 |
| (a) Neo Heliopan ® BB | 1.0 |
| (a) Copherol ® F 1300 | 1.0 |
| (b) Glycerol, 86% | 5.0 |
| (b) Water | to 100 |

²⁵3-Methylbutyl-3-(4-methoxyphenyl)-2-propenoate) (INCI name: Isoamyl-p-methoxycinnamate) (HAARMANN & REIMER)

Preparation, as Example 1.

1.-12. (Cancelled)

13. A composition for treating keratin fibers in the form of a microemulsion with a mean droplet diameter of less than 400 nanometers, comprising at least 6% by weight of an oil component, at least one nonionic emulsifier, and a UV-absorbing component A corresponding to general formula (I):
U−Q (I)
in which U is a UV-absorbing group and Q is a group which contains at least one quaternary ammonium function.

14. The composition of claim 13, wherein the group Q in formula (I) has the general structure —(CH₂)ₓ—N⁺R¹R²R³ X⁻, in which x is an integer of 1 to 4, R¹ and R² independently of one another represent C₁₋₄ alkyl groups, R³ is a C₁₋₂₂ alkyl group or a benzyl group and X⁻ is a physiologically compatible anionic group.

15. The composition of claim 14, wherein at least two of the groups R¹, R² and R³ are methyl groups.

16. The composition of claim 13, wherein the group U is selected so that the compound A has an absorption maximum in the UVB range.

17. The composition of claim 14, wherein the group U is selected so that the compound A has an absorption maximum in the UVB range.

18. The composition of claim 16, wherein the compound A has a molar extinction coefficient at the absorption maximum of at least 15,000.

19. The composition of claim 13, comprising at least one oil component of vegetable origin.

20. The composition of claim 13, comprising at least one nonionic emulsifier with an HLB value of 8 to 18.

21. The composition of claim 13, comprising at least one ethoxylated fatty alcohol as a nonionic emulsifier.

22. The composition of claim 13, comprising at least one additional hair-care component.

23. The composition of claim 13, comprising at least one other UV filter component.

24. The composition of claim 13, in the form of a sprayable composition.

25. A process for treating keratin fibers, comprising the steps of contacting a keratin fiber with the composition of claim 13 for a contact time of about 1 second to about 30 minutes, and subsequently rinsing said composition from the keratin fiber.

26. A composition for treating keratin fibers in the form of a microemulsion with a mean droplet diameter of less than 400 nanometers comprising at least 6% by weight of an oil component, at least one nonionic emulsifier and a UV absorbing component: dodecyl dimethyl aminobenzamidopropyl dimethyl ammonium tosylate.
